(19) United States
(12) Patent Application Publication
(10) Pub. No.: US 2008/0215250 A1
(43) Pub. Date: Sep. 4, 2008

(54) Molecular toxicology modeling

(76) Inventors: Donna L. Mendrick, Mount Airy, MD (US); Mark W. Porter, Germantown, MD (US); Kory R. Johnson, Bethesda, MD (US); Arthur L. Castle, Washington, DC (US); Michael R. Elashoff, Germantown, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/790,979

(22) Filed: Apr. 30, 2007

Related U.S. Application Data
(63) Continuation of application No. 09/917,800, filed on Jul. 31, 2001, now abandoned.
(60) Provisional application No. 60/222,040, filed on Jul. 31, 2000.
(60) Provisional application No. 60/244,880, filed on Nov. 2, 2000.
(60) Provisional application No. 60/290,029, filed on May 11, 2001.
(60) Provisional application No. 60/290,645, filed on May 15, 2001.
(60) Provisional application No. 60/292,336, filed on May 22, 2001.
(60) Provisional application No. 60/295,798, filed on Jun. 6, 2001.
(60) Provisional application No. 60/297,457, filed on Jun. 13, 2001.
(60) Provisional application No. 60/298,884, filed on Jun. 19, 2001.
(60) Provisional application No. 60/303,459, filed on Jul. 9, 2001.

(51) Int. Cl. G01N 33/48

(52) U.S. Cl. 702/20

(57) ABSTRACT

The present invention is based on the elucidation of the global changes in gene expression and the identification of toxicity markers in tissues or cells exposed to a known toxin. The genes may be used as toxicity markers in drug screening and toxicity assays. The invention includes a database of genes characterized by toxin-induced differential expression that is designed for use with microarrays and other solid-phase probes.

### RELATED APPLICATIONS

This application is related to U.S. Provisional Applications 60/222,040, 60/244,880, 60/290,029, 60/290,645, 60/292,336, 60/295,798, 60/297,457, 60/298,884 and 60/303,459, all of which are herein incorporated by reference in their entirety.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are most easily maintained and manipulated. Unicellular screening systems also often use easily detectable changes in phenotype to monitor the effect of test compounds on the cell. Unicellular organisms, however, are inadequate models for estimating the potential effects of many compounds on complex multicellular animals, as they do not have the ability to carry out biotransformations to the extent or at levels found in higher organisms.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. In addition, previous attempts to produce toxicology prediction systems have failed to provide the necessary modeling information (e.g. WO0012760, WO0047761, WO0063435, WO0132928A2, WO0138579A2, and the Affymetrix® Rat Tox Chip.

### SUMMARY OF THE INVENTION

The present invention is based on the elucidation of the global changes in gene expression in tissues or cells exposed to known toxins, in particular hepatotoxins, as compared to unexposed tissues or cells as well as the identification of individual genes that are differentially expressed upon toxin exposure.

In various aspects, the invention includes methods of predicting at least one toxic effect of a compound, predicting the progression of a toxic effect of a compound, and predicting the hepatoxicity of a compound. The invention also includes methods of identifying agents that modulate the onset or progression of a toxic response. Also provided are methods of predicting the cellular pathways that a compound modulates in a cell. The invention includes methods of identifying agents that modulate protein activities.

In a further aspect, the invention provides probes comprising sequences that specifically hybridize to genes in Tables 1-3. Also provided are solid supports comprising at least two of the previously mentioned probes. The invention also includes a computer system that has a database containing information identifying the expression level in a tissue or cell sample exposed to a hepatotoxin of a set of genes comprising at least two genes in Tables 1-3.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cells. For example, the lack of sufficient expression of functional tumor suppressor genes and/or the over expression of oncogene/protooncogenes after exposure to an agent could lead to tumorgenesis or hyperplastic growth of cells (Marshall, Cell, 64: 313-326 (1991); Weinberg, Science, 254:1138-1146 (1991)). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to the known hepatotoxins which induce detrimental liver effects, to identify global changes in gene expression induced by these compounds. These global changes in gene expression, which can be detected by the production of expression profiles, provide useful toxicity markers that can be used to monitor toxicity and/or toxicity progression by a test compound. Some of these markers may also be used to monitor or detect various disease or physiological states, disease progression, drug efficacy and drug metabolism.

### Identification of Toxicity Markers

To evaluate and identify gene expression changes that are predictive of toxicity, studies using selected compounds with well characterized toxicity have been conducted by the present inventors to catalogue altered gene expression during exposure in vivo and in vitro. In the present study, amitryptiline, alpha-naphthylisothiocyante (ANIT), acetaminophen, carbon tetrachloride, cyproterone acetate (CPA), diclofenac, 17α-ethinylestradiol, indomethacin, valproate and WY-14643 were selected as a known hepatotoxins.

The pathogenesis of acute CCl₄-induced hepatotoxicity follows a well-characterized course in humans and experimental animals resulting in centrilobular necrosis and steatosis, followed by hepatic regeneration and tissue repair. Severity of the hepatocellular injury is also dose-dependent and may be affected by species, age, gender and diet.

Differences in susceptibility to CCl₄ hepatotoxicity are primarily related to the ability of the animal model to metabolize CCl₄ to reactive intermediates. CCl₄-induced hepatotoxicity is dependent on CCl₄ bioactivation to trichloromethyl free radicals by cytochrome P450 enzymes (CYP2E1), localized primarily in centrizonal hepatocytes. Formation of the free radicals leads to membrane lipid peroxidation and protein denaturation resulting in hepatocellular damage or death.

The onset of hepatic injury is rapid following acute administration of CCl₄ to male rats. Morphologic studies have shown cytoplasmic accumulation of lipids in hepatocytes within 1 to 3 hours of dosing, and by 5 to 6 hours, focal necrosis and hydropic swelling of hepatocytes are evident. Centrilobular necrosis and inflammatory infiltration peak by 24 to 48 hours post dose. The onset of recovery is also evident within this time frame by increased DNA synthesis and the appearance of mitotic figures. Removal of necrotic debris begins by 48 hours and is usually completed by one week, with full restoration of the liver by 14 days.

Increases in serum transaminase levels also parallel CCl₄-induced hepatic histopathology. In male Sprague Dawley (SD) rats, alanine aminotrasferase (ALT) and aspartate aminotransferase (AST) levels increase within 3 hours of CCl₄ administration (0.1, 1, 2, 3, 4 mL/kg, ip; 2.5 mL/kg, po) and reach peak levels (approximately 5-10 fold increases) within 48 hours post dose. Significant increases in serum α-glutathione s-transferase (α-GST) levels have also been detected as early as 2 hours after CCl₄ administration (25 μL/kg, po) to male SD rats.

At the molecular level, induction of the growth-related proto-oncogenes, c-fos and c-jun, is reportedly the earliest event detected in an acute model of CCl₄-induced hepatotoxicity (Schiaffonato et al. (1997) Liver 17:183-191). Expression of these early-immediate response genes has been detected within 30 minutes of a single dose of CCl₄ to mice (0.05-1.5 mL/kg, ip) and by 1 to 2 hours post dose in rats (2 mL/kg, po; 5 mL/kg,po) (Schiaffonato et al. (1997) Liver 17:183-191 and Hong et al. (1997) Yonsei Medical. J. 38:167-177). Similarly, hepatic c-myc gene expression is increased by 1 hour following an acute dose of CCl₄ to male SD rats (5 mL/kg, po) (Hong et al.). Expression of these genes following exposure to CCl₄ is rapid and transient. Peak hepatic mRNA levels for c-fos, c-jun, and c-myc, after acute administration of CCl₄ have been reported at 1 to 2 hours, 3 hours, and 1 hour post dose, respectively.

The expression of tumor necrosis factor-α (TNF-α) is also increased in the livers of rodents exposed to CCl₄, and TNF-α has been implicated in initiation of the hepatic repair process. Pre-treatment with anti-TNF-α antibodies has been shown to prevent CCl₄-mediated increases in c-jun and c-fos gene expression, whereas administration of TNF-α induced rapid expression of these genes (Bruccoleri et al. (1997) Hepatol. 25:133-141). Up-regulation of transforming growth factor-β (TGF-β) and transforming growth factor receptors (TBRI-III) later in the repair process (24 and 48 hours after CCl₄ administration) suggests that TGF-β may play a role in limiting the regenerative response by induction of apoptosis. (Grasl-Kraupp et al. (19.98) Hepatol. 28:717-7126).

Acetaminophen is a widely used analgesic that at supratherapeutic doses can be metabolized to N-acetyl-p-benzoquinone imine (NAPQI) which causes hepatic and renal failure. At the molecular level, until the present invention little was known about the effects of acetominophen.

Amitriptyline is a commonly used antidepressant, although it is recognized to have toxic effects on the liver (Physicians Desk Reference, 47^{th} ed., Medical Economics Co., Inc., 1993; Balkin, U.S. Pat. No. 5,656,284). Nevertheless, amitriptyline's beneficial effects on depression, as well as on sleep and dyspepsia (H. Mertz et al., Am J Gastroenterol 93(2):160-165, 1998), migraines (E. Beubler, Wien Med Wochenschr 144(5-6):100-101, 1994), arterial hypertension (T. Bobkiewicz et al., Arch Immunol Ther Exp (Warsz) 23(4):543-547, 1975) and premature ejaculation (Smith et al., U.S. Pat. No. 5,923,341) mandate its continued use.

Differences in susceptibility to amitriptyline toxicity are considered related to differential metabolism. Amitriptyline-induced hepatotoxicity is primarily mediated by development of cholestasis, the condition caused by the failure of the liver to secrete bile, resulting in accumulation in blood plasma of substances normally secreted into bile-bilirubin and bile salts. Cholestasis is also characterized by liver cell necrosis and bile duct obstruction, which leads to increased pressure on the lumenal side of the canalicular membrane and release of enzymes (alkaline phosphatase, 5′-nucleotidase, gammaglutamyl transpeptidase) normally localized on the canalicular membrane. These enzymes also begin to accumulate in the plasma. Typical symptoms of cholestasis are general malaise, weakness, nausea, anorexia and severe pruritis (Cecil Textbook of Medicine, 20^{th} ed., part XII, pp. 772-773, 805-808, J. C. Bennett and F. Plum Eds., W. B. Saunders Co., Philadelphia, 1996).

The effects of amitriptyline or phenobarbital (PB) on phospholipid metabolism in rat liver have been studied. In one study, male Sprague-Dawley rats received amitriptyline orally in one dose of 600 mg/kg. PB was given intraperitonially (IP) at a dosage of 80 mg/kg. Animals were sacrificed by decapitation at 6, 12, 18, and 24 hr. The phospholipid level in liver was measured by enzymatic assay and by gas chromatography-mass spectrometry. Both agents caused an increase in the microsomal phosphatidylcholine content. Levels of glycerophosphate acyltransferase (GAT) and phosphatidate cytidylyltransferase (PCT) were slightly affected by amitriptyline but were significantly affected by PB. Levels of phosphatidate phosphohydrolase (PPH) and choline phosphotransferase (CPT) were significantly altered by amitriptyline and by PB (K. Hoshi et al., “Effect of amitriptyline or phenobarbital on the activities of the enzymes involved in rat liver,” Chem Pharm Bull 38:3446-3448, 1990).

In another experiment, amitriptyline was given orally to male Sprague-Dawley rats (4-5 weeks old) in a single dose of 600 mg/kg. The animals were sacrificed 12 or 24 hours later. This caused a marked increase in δ-aminolevulinic acid (δ-ALA) activity at both time points. Total heme and cytochrome b5 levels were increased but cytochrome P450 (CYP450) content remained the same. The authors concluded that hepatic heme synthesis is increased through prolonged induction of δ-ALA but this may be accounted for by the increases in cytochrome b5 and total heme and not by the CYP450 content (K. Hoshi et al., “Acute effect of amitriptyline, phenobarbital or cobaltous chloride on δ-aminolevulinic acid synthetase, heme oxygenase and microsomal heme content and drug metabolism in rat liver”, Jpn J Pharmacol 50:289-293, 1989).

Amitriptyline can cause hypersensititivity syndrome, a specific severe idiosyncratic reaction characterized by skin, liver, joint and haematological abnormalities (H. J. Milionis et al., Postgrad Med 76(896):361-363, 2000). Amitriptyline has also been shown to cause drug-induced hepatitis, resulting in liver peroxisomes with impaired catalase function (D. De Creaemer et al., Hepatology 14(5):811-817, 1991). The peroxisomes are larger in number, but smaller in size and deformed in shape. Using cultured hepatocytes, the cytotoxicity of amitriptyline was examined and compared to other psychotropic drugs (U. A. Boelsterli et al., Cell Biol Toxicol 3(3):231-250, 1987). The effects observed were release of lactate dehydrogenase from the cytosol, as well as impairment of biosynthesis and secretion of proteins, bile acids and glycolipids.

Aromatic and aliphatic isothiocyanates are commonly used soil fumigants and pesticides (E. Shaaya et al., Pesticide Science 44(3):249-253, 1995; T. Cairns et al., J Assoc Official Analytical Chemists 71(3):547-550, 1988). These compounds are also environmental hazards, however, because they remain as toxic residues in plants, either in their original or in a metabolized form (M. S. Cerny et al., J Agricultural and Food Chemistry 44(12):3835-3839, 1996) and because they are released from the soil into the surrounding air (J. Gan et al., J Agricultural and Food Chemistry 46(3):986-990, 1998). Alpha-naphthylthiourea, an amino-substituted form of ANIT, is a known rodenticide whose principal toxic effects are pulmonary edema and pleural effusion, resulting from the action of this compound on pulmonary capillaries. Microsomes from lung and liver release atomic sulfur (Goodman and Gilman's The Pharmacological Basis of Therapeutics, 9^{th} ed., chapter 67, p. 1690, J. G. Hardman et al. Eds., McGraw-Hill, New York, N.Y., 1996).

In one study in rats, ANIT (80 mg/kg) was dissolved in olive oil and given orally to male Wistar rats (180-320 g). All animals were fasted for 24 hours before ANIT treatment, and blood and bile excretion were analyzed 24 hours later. Levels of total bilirubin, alkaline phosphatase, serum glutamic oxaloacetic transaminase and serum glutamic pyruvic transaminase were found to be significantly increased, while ANIT reduced total bile flow, all of which are indications of severe biliary dysfunction. This model is used to induce cholestasis with jaundice because the injury is reproducible and dose-dependent. ANIT is metabolized by microsomal enzymes, and a metabolite plays a fundamental role in its toxicity (M. Tanaka et al., “The inhibitory effect of SA3443, a novel cyclic disulfide compound, on alpha-naphthyl isothiocyanate-induced intrahepatic cholestasis in rats,” Clinical and Experimental Pharmacology and Physiology 20:543-547, 1993).

ANIT fails to produce extensive necrosis, but has been found to produce inflammation and edema in the portal tract of the liver (T. J. Maziasa et al., “The differential effects of hepatotoxicants on the sulfation pathway in rats,” Toxicol Appl Pharmacol 110:365-373, 1991). Livers treated with ANIT are significantly heavier than control-treated counterparts and serum levels of alanine aminotransferase (ALT), gamma-glutamyl transpeptidase (γ-GTP), total bilirubin, lipid peroxide and total bile acids showed significant increases (Anonymous, “An association between lipid peroxidation and α-naphthylisothiocyanate-induced liver injury in rats,” Toxicol Lett 105:103-110, 2000).

ANIT-induced hepatotoxicity may also be characterized by cholangiolitic hepatitis and bile duct damage. Acute hepatotoxicity caused by ANIT in rats is manifested as neutrophil-dependent necrosis of bile duct epithelial cells (BDECs) and hepatic parenchymal cells. These changes mirror the cholangiolitic hepatitis found in humans (D. A. Hill, Toxicol Sci 47:118-125, 1999).

Exposure to ANIT also causes liver injury by the development of cholestasis, the condition caused by failure to secrete bile, resulting in accumulation in blood plasma of substances normally secreted into bile, such as bilirubin and bile salts. Cholestasis is also characterized by liver cell necrosis, including bile duct epithelial cell necrosis, and bile duct obstruction, which leads to increased pressure on the lumenal side of the canalicular membrane, decreased canalicular flow and release of enzymes normally localized on the canalicular membrane (alkaline phosphatase, 5′-nucleotidase, gammaglutamyl transpeptidase). These enzymes also begin to accumulate in the plasma. Typical symptoms of cholestasis are general malaise, weakness, nausea, anorexia and severe pruritis (Cecil Textbook of Medicine, 20^{th} ed., part XII, pp. 772-773, 805-808, J. C. Bennett and F. Plum Eds., W. B. Saunders Co., Philadelphia, 1996 and D. C. Kossor et al., “Temporal relationship of changes in hepatobiliary function and morphology in rats following α-naphthylisothiocyanate (ANIT) administration,” Toxicol Appl Pharmacol 119:108-114, 1993).

ANIT-induced cholestatis is also characterized by abnormal serum levels of alanine aminotransferase, aspartic acid aminotransferase and total bilirubin. In addition, hepatic lipid peroxidation is increased, and the membrane fluidity of microsomes is decreased. Histological changes include an infiltration of polymorphonuclear neutrophils and elevated number of apoptotic hepatocytes (J. R. Calvo et al., J Cell Biochem 80(4):461-470, 2001). Other known hepatotoxic effects of exposure to ANIT include a damaged antioxidant defense system, decreased activities of superoxide dismutase and catalase (Y. Ohta et al. Toxicology 139(3):265-275, 1999), and the release of several proteases from the infiltrated neutrophils, alanine aminotransferase, cathepsin G, elastase, which mediate hepatocyte killing (D. A. Hill et al., Toxicol Appl Pharmacol 148(1):169-175, 1998).

Indomethacin is a non-steroidal antiinflammatory, antipyretic and analgesic drug commonly used to treat rheumatoid arthritis, osteoarthritis, ankylosing spondylitis, gout and a type of severe, chronic cluster headache characterized by many daily occurrences and jabbing pain. This drug acts as a potent inhibitor of prostaglandin synthesis; it inhibits the cyclooxygenase enzyme necessary for the conversion of arachidonic acid to prostaglandins (PDR 47^{th} ed., Medical Economics Co., Inc., Montvale, N.J., 1993; Goodman & Gilman's The Pharmalogical Basis of Therapeutics 9^{th} ed., J. G. Hardman et al. Eds., McGraw Hill, New York, 1996, pp. 1074-1075, 1089-1095; Cecil Textbook of Medicine, 20^{th} ed., part XII, pp. 772-773, 805-808, J. C. Bennett and F. Plum Eds., W. B. Saunders Co., Philadelphia, 1996).

The most frequent adverse effects of indomethacin treatment are gastrointestinal disturbances, usually mild dyspepsia, although more severe conditions, such as bleeding, ulcers and perforations can occur. Hepatic involvement is uncommon, although some fatal cases of hepatitis and jaundice have been reported. Renal toxicity can also result, particularly after long-term administration. Renal papillary necrosis has been observed in rats, and interstitial nephritis with hematuria, proteinuria and nephrotic syndrome have been reported in humans. Patients suffering from renal dysfunction risk developing a reduction in renal blood flow, because renal prostaglandins play an important role in renal perfusion.

In rats, although indomethacin produces more adverse effects in the gastrointestinal tract than in the liver, it has been shown to induce changes in hepatocytic cytochrome P450. In one study, no widespread changes in the liver were observed, but a mild, focal, centrilobular response was noted. Serum levels of albumin and total protein were significantly reduced, while the serum level of urea was increased. No changes in creatinine or aspartate aminotransferase (AST) levels were observed (M. Falzon et al., “Comparative effects of indomethacin on hepatic enzymes and histology and on serum indices of liver and kidney function in the rat,” Br J exp Path 66:527-534, 1985). In another rat study, a single dose of indomethacin has been shown to reduce liver and renal microsomal enzymes, including CYP450, within 24 hours. Histopathological changes were not monitored, although there were lesions in the GI tract. The effects on the liver seemed to be waning by 48 hours (M. E. Fracasso et al. “Indomethacin induced hepatic alterations in mono-oxygenase system and faecal clostridium perfringens enterotoxin in the rat,” Agents Actions 31:313-316, 1990).

A study of hepatocytes, in which the relative toxicity of five nonsteroidal antiinflammatory agents was compared, showed that indomethacin was more toxic than the others. Levels of lactate dehydrogenase release and urea, as well as viability and morphology, were examined. Cells exposed to high levels of indomethacin showed cellular necrosis, nuclear pleomorphism, swollen mitochondria, fewer microvilli, smooth endoplasmic reticulum proliferation and cytoplasmic vacuolation (E. M. Sorensen et al., “Relative toxicities of several nonsteroidal antiinflammatory compounds in primary cultures of rat hepatocytes,” J Toxicol Environ Health 16(3-4); 425-440, 1985).

17α-ethinylestradiol, a synthetic estrogen, is a component of oral contraceptives, often combined with the progestational compound norethindrone. It is also used in post-menopausal estrogen replacement therapy (PDR 47^{th} ed., pp. 2415-2420, Medical Economics Co., Inc., Montvale, N.J., 1993; Goodman & Gilman's The Pharmalogical Basis of Therapeutics 9^{th} ed., pp. 1419-1422, J. G. Hardman et al. Eds., McGraw Hill, New York, 1996).

The most frequent adverse effects of 17α-ethinylestradiol usage are increased risks of cardiovascular disease: myocardial infarction, thromboembolism, vascular disease and high blood pressure, and of changes in carbohydrate metabolism, in particular, glucose intolerance and impaired insulin secretion. There is also an increased risk of developing benign hepatic neoplasia, although the incidence of this disease is very low. Because this drug decreases the rate of liver metabolism, it is cleared slowly from the liver, and carcinogenic effects, such as tumor growth, may result.

In a recent study, 17α-ethinylestradiol was shown to cause a reversible intrahepatic cholestasis in male rats, mainly by reducing the bile-salt-independent fraction of bile flow (BSIF) (N. R. Koopen et al., “Impaired activity of the bile canalicular organic anion transporter (Mrp2/cmoat) is not the main cause of ethinylestradiol-induced cholestasis in the rat,” Hepatology 27:537-545, 1998). Plasma levels of bilirubin, bile salts, aspartate aminotransferase (AST) and alanine aminotransferase (ALT) in this study were not changed. This study also showed that 17α-ethinylestradiol produced a decrease in plasma cholesterol and plasma triglyceride levels, but an increase in the weight of the liver after 3 days of drug administration, along with a decrease in bile flow. Further results from this study are as follows. The activities of the liver enzymes leucine aminopeptidase and alkaline phosphatase initially showed significant increases, but enzyme levels decreased after 3 days. Bilirubin output increased, although glutathione (GSH) output decreased. The increased secretion of bilirubin into the bile without affecting the plasma level suggests that the increased bilirubin production must be related to an increased degradation of heme from heme-containing proteins. Similar results were obtained in another experiment (G. Bouchard et al., “Influence of oral treatment with ursodeoxycholic and tauroursodeoxycholic acids on estrogen-induced cholestasis in rats: effects on bile formation and liver plasma membranes,” Liver 13:193-202, 1993) in which the livers were also examined by light and electron microscopy. Despite the effects of the drug, visible changes in liver tissue were not observed.

In another study of male rats, cholestasis was induced by daily subcutaneous injections of 17α-ethinylestradiol for five days. Cholestasis was assessed by measuring the bile flow rate. Rats allowed to recover for five days after the end of drug treatment showed normal bile flow rates (Y. Hamada et al., “Hormone-induced bile flow and hepatobiliary calcium fluxes are attenuated in the perfused liver of rats made cholestatic with ethynylestradiol in vivo and with phalloidin in vitro,” Hepatology 21:1455-1464, 1995).

An experiment with male and female rats (X. Mayol, “Ethinyl estradiol-induced cell proliferation in rat liver. Involvement of specific populations of hepatocytes,” Carcinogenesis 13:2381-2388, 1992) found that 17α-ethinylestradiol induced acute liver hyperplasia (increase in mitotic index and BrdU staining) after two days of treatment, although growth regression occurred within the first few days of treatment. With long-term treatment, lasting hyperplasia was again observed after three to six months of administration of the drug. Apoptosis increased around day 3 and returned to normal by one week. Additional experiments in this same study showed that proliferating hepatocytes were predominantly located around a periportal zone of vacuolated hepatocytes, which were also induced by the treatment. Chronic induced activation was characterized by flow cytometry on hepatocytes isolated from male rats, and ploidy analysis of hepatocyte cell suspensions showed a considerably increased proportion of diploid hepatocytes. These diploid cells were the most susceptible to drug-induced proliferation. The results from this study support the theory that cell target populations exist that respond to the effects of tumor promoters. The susceptibility of the diploid hepatocytes to proliferation during treatment may explain, at least in part, the behavior of 17α-ethinylestradiol as a tumor promoter in the liver.

Wy-14643, a tumor-inducing compound that acts in the liver, has been used to study the genetic profile of cells during the various stages of carcinogenic development, with a view toward developing strategies for detecting, diagnosing and treating cancers (J. C. Rockett et al., “Use of suppression-PCR subtractive hybridisation to identify genes that demonstrate altered expression in male rat and guinea pig livers following exposure to Wy-14,643, a peroxisome proliferator and non-genotoxic hepatocarcinogen,” Toxicology 144(1-3):13-29, 2000). In contrast to other carcinogens, Wy-14643 does not mutate DNA directly. Instead, it acts on the peroxisome proliferator activated receptor-alpha (PPARalpha), as well as on other signaling pathways that regulate growth (T. E. Johnson et al., “Peroxisome proliferators and fatty acids negatively regulate liver X receptor-mediated activity and sterol biosynthesis,” J Steroid Biochem Mol Biol. 77(1):59-71, 2001). The effect is elevated and sustained cell replication, accompanied by a decrease in apoptosis (I. Rusyn et al., “Expression of base excision repair enzymes in rat and mouse liver is induced by peroxisome proliferators and is dependent upon carcinogenic potency,” Carcinogenesis 21(12):2141-2145, 2000). These authors (Rusyn et al.) noted an increase in the expression of enzymes that repair DNA by base excision, but no increased expression of enzymes that do not repair oxidative damage to DNA. In a study on rodents, Johnson et al. noted that Wy-14643 inhibited liver-X-receptor-mediated transcription in a dose-dependent manner, as well as de novo sterol synthesis.

In experiments with mouse liver cells (J. M. Peters et al., “Role of peroxisome proliferator-activated receptor alpha in altered cell cycle regulation in mouse liver,” Carcinogenesis 19(11): 1989-1994, 1998), exposure to Wy-14643 produced increased levels of acyl CoA oxidase and proteins involved in cell proliferation: CDK-1, 2 and 4, PCNA and c-myc. Elevated levels may be caused by accelerated transcription that is mediated directly or indirectly by PPARalpha. It is likely that the carcinogenic properties of peroxisome proliferators are due to the PPARalpha-dependent changes in levels of cell cycle regulatory proteins.

Another study on rodents (B. J. Keller et al., “Several nongenotoxic carcinogens uncouple mitochondrial oxidative phosphorylation,” Biochim Biophys Acta 1102(2):237-244, 1992) showed that Wy-14643 was capable of uncoupling oxidative phosphorylation in rat liver mitochondria. Rates of urea synthesis from ammonia and bile flow, two energy-dependent processes, were reduced, indicating that the energy supply for these processes was disrupted as a result of cellular exposure to the toxin.

Wy-14643 has also been shown to activate nuclear factor kappaB, NADPH oxidase and superoxide production in Kupffer cells (I. Rusyn et al., “Oxidants from nicotinamide adenine dinucleotide phosphate oxidase are involved in triggering cell proliferation in the liver due to peroxisome proliferators,” Cancer Res 60(17):4798-4803, 2000). NADPH oxidase is known to induce mitogens, which cause proliferation of liver cells.

CPA is a potent androgen antagonist and has been used to treat acne, male pattern baldness, precocious puberty, and prostatic hyperplasia and carcinoma (Goodman & Gilman's The Pharmacological Basis of Therapeutics 9^{th} ed., p. 1453, J. G. Hardman et al., Eds., McGraw Hill, New York, 1996). Additionally, CPA has been used clinically in hormone replacement therapy (HRT). CPA is useful in HRT as it protects the endometrium, decreases menopausal symptoms, and lessens osteoporotic fracture risk (H. P. Schneider, “The role of antiandrogens in hormone replacement therapy,” Climacteric 3 (Suppl. 2): 21-27, 2000).

Although CPA has numerous clinical applications, it is tumorigenic, mitogenic, and mutagenic. CPA has been used to treat patients with adenocarcinoma of the prostate, however in two documented cases (A. G. Macdonald and J. D. Bissett, “Avascular necrosis of the femoral head in patients with prostate cancer treated with cyproterone acetate and radiotherapy,” Clin Oncol 13: 135-137, 2001), patients developed femoral head avascular necrosis following CPA treatment. In one study (O. Krebs et al., “The DNA damaging drug cyproterone acetate causes gene mutations and induces glutathione-S-transferase P in the liver of female Big Blue transgenic F344 rats,” Carcinogenesis 19(2): 241-245, 1998), Big Blue transgenic F344 rats were giving varying doses of CPA. As the dose of CPA increased, so did the mutation frequency, but a threshold dose was not determined. Another study (S. Werner et al., “Formation of DNA adducts by cyproterone acetate and some structural analogues in primary cultures of human hepatocytes,” Mutat Res 395(2-3): 179-187, 1997), showed that CPA caused the formation of DNA adducts in primary cultures of human hepatocytes. The authors suggest that the genotoxicity associated with CPA may be due to the double bond in position 6-7 of the steroid.

In additional experiments with rats (P. Kasper and L. Mueller, “Time-related induction of DNA repair synthesis in rat hepatocytes following in vivo treatment with cyproterone acetate,” Carcinogenesis 17(10): 2271-2274, 1996), CPA was shown to induce unscheduled DNA synthesis in vitro. After a single oral dose of 100 mg CPA/kg body weight, continuous DNA repair activity was observed after 16 hours. Furthermore, CPA increased the occurrence of S phase cells, which corroborated the mitogenic potential of CPA in rat liver.

CPA has also been shown to produce cirrhosis (B. Z. Garty et al., “Cirrhosis in a child with hypothalamic syndrome and central precocious puberty treated with cyproterone acetate,” Eur J Pediatr 158(5): 367-370, 1999). A child, who had been treated with CPA for over 4 years for hypothalamic syndrome and precocious puberty, developed cirrhosis. Even though the medication was discontinued, the child eventually succumbed to sepsis and multiorgan failure four years later.

In one study on rat liver treated with CPA (W. Bursch et al., “Expression of clusterin (testosterone-repressed prostate message-2) mRNA during growth and regeneration of rat liver,” Arch Toxicol 69(4): 253-258, 1995), the expression of clusterin, a marker for apoptosis, was examined and measured by Northern and slot blot analysis. Bursch et al. showed that post-CPA administration, the clusterin mRNA concentration level increased. Moreover, in situ hybridization demonstrated that clusterin was expressed in all hepatocytes, therefore it is not limited to cells in the process of death by apoptosis.

Diclofenac, a non-steroidal anti-inflammatory drug, has been frequently administered to patients suffering from rheumatoid arthritis, osteoarthritis, and ankylosing spondylitis. Following oral administration, diclofenac is rapidly absorbed and then metabolized in the liver by cytochrome P450 isozyme of the CYC2C subfamily (Goodman & Gilman's The Pharmacological Basis of Therapeutics 9^{th} ed., p. 637, J. G. Hardman et al., Eds., McGraw Hill, New York, 1996). In addition, diclofenac has been applied topically to treat pain due to corneal damage (D. G. Jayamanne et al., “The effectiveness of topical diclofenac in relieving discomfort following traumatic corneal abrasions,” Eye 11(Pt. 1): 79-83, 1997; D. I. Dornic et al., “Topical diclofenac sodium in the management of anesthetic abuse keratopathy,” Am J Opthalmol 125(5): 719-721, 1998).

Although diclofenac has numerous clinical applications, adverse side-effects have been associated with the drug. In one study, out of 16 patients suffering from corneal complications associated with diclofenac use, 6 experienced corneal or scleral melts, three experienced ulceration, and two experienced severe keratopathy (A. C. Guidera et al., “Keratitis, ulceration, and perforation associated with topical nonsteroidal anti-inflammatory drugs,” Opthalmology 108(5): 936-944, 2001). Another report described a term newborn who had premature closure of the ductus arteriosus as a result of maternal treatment with diclofenac (M. Zenker et al., “Severe pulmonary hypertension in a neonate caused by premature closure of the ductus arteriosus following maternal treatment with diclofenac: a case report,” J Perinat Med 26(3): 231-234, 1998). Although it was only two weeks prior to delivery, the newborn had severe pulmonary hypertension and required treatment for 22 days of high doses of inhaled nitric oxide.

Another study investigated 180 cases of patients who had reported adverse reactions to diclofenac to the Food and Drug Administration (A. T. Banks et al., “Diclofenac-associated hepatoxicity: analysis of 180 cases reported to the Food and Drug Administration as adverse reactions,” Hepatology 22(3): 820-827, 1995). Of the 180 reported cases, the most common symptom was jaundice (75% of the symptomatic patients). Liver sections were taken and analyzed, and hepatic injury was apparent one month after drug treatment. An additional report showed that a patient developed severe hepatitis five weeks after beginning diclofenac treatment for osteoarthritis (A. Bhogaraju et al., “Diclofenac-associated hepatitis,” South Med J 92(7): 711-713, 1999). Within a few months following the cessation of diclofenac treatment there was complete restoration of liver functions.

In one study on diclofenac-treated Wistar rats (P. E. Ebong et al., “Effects of aspirin (acetylsalicylic acid) and Cataflam (potassium diclofenac) on some biochemical parameters in rats,” Afr J Med Med Sci 27(3-4): 243-246, 1998), diclofenac treatment induced an increase in serum chemistry levels of alanine aminotransferase, aspartate aminotransferase, methaemoglobin, and total and conjugated bilirubin. Additionally, diclofenac enhanced the activity of alkaline phosphatase and 5′nucleotidase. Another study showed that humans given diclofenac had elevated levels of hepatic transaminases and serum creatine when compared to the control group (F. McKenna et al., “Celecoxib versus diclofenac in the management of osteoarthritis of the knee,” Scand J Rheumatol 30(1): 11-18, 2001).

### Toxicity Prediction and Modeling

The genes and gene expression information, as well as the portfolios and subsets of the genes provided in Tables 1-3, may be used to predict at least one toxic effect, including the hepatotoxicity of a test or unknown compound. As used, herein, at least one toxic effect includes, but is not limited to, a detrimental change in the physiological status of a cell or organism. The response may be, but is not required to be, associated with a particular pathology, such as tissue necrosis. Accordingly, the toxic effect includes effects at the molecular and cellular level. Hepatotoxicity is an effect as used herein and includes but is not limited to the pathologies of liver necrosis, hepatitis, fatty liver and protein adduct formation.

In general, assays to predict the toxicity or hepatotoxicity of a test agent (or compound or multi-component composition) comprise the steps of exposing a cell population to the test compound, assaying or measuring the level of relative or absolute gene expression of one or more of the genes in Tables 1-3 and comparing the identified expression level(s) to the expression levels disclosed in the Tables and database(s) disclosed herein. Assays may include the measurement of the expression levels of about 2, 3, 4, 5, 6, 7, 8, 9, 10, 15, 20, 25, 30, 50, 75, 100 or more genes from Tables 1-3.

In the methods of the invention, the gene expression level for a gene or genes induced by the test agent, compound or compositions may be comparable to the levels found in the Tables or databases disclosed herein if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated hepatocytes, in particular rat hepatocytes, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al. Loomis's Essentials of Toxicology, 4th Ed. (Academic Press, New York, 1996); Echobichon, The Basics of Toxicity Testing (CRC Press, Boca Raton, 1992); Frazier, editor, In Vitro Toxicity Testing (Marcel Dekker, New York, 1992); and the like.

In in vitro toxicity testing, two groups of test organisms are usually employed: One group serves as a control and the other group receives the test compound in a single dose (for acute toxicity tests) or a regimen of doses (for prolonged or chronic toxicity tests). Since in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration. dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from liver tissue. For instance, cultured or freshly isolated rat hepatocytes may be used.

The methods of the invention may be used to generally predict at least one toxic response, and as described in the Examples, may be used to predict the likelihood that a compound or test agent will induce various specific liver pathologies such as liver necrosis, fatty liver disease, protein adduct formation or hepatitis. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin (see Tables 3A-3S).

### Diagnostic Uses for the Toxicity Markers

As described above, the genes and gene expression information or portfolios of the genes with their expression information as provided in Tables 1-3 may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes from Tables 1-3 may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

In another format, the levels of a gene(s) of Tables 1-3, its encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

### Use of the Markers for Monitoring Toxicity Progression

As described above, the genes and gene expression information provided in Tables 1-3 may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes from Tables 1-3 may be compared to the expression levels found in tissue or cells exposed to the hepatotoxins described herein. The comparison of the expression data, as well as available sequence or other information may be done by researcher or diagnostician or may be done with the aid of a computer and databases.

### Use of the Toxicity Markers for Drug Screening

According to the present invention, the genes identified in Tables 1-3 may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to simulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1-3 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, tow or more genes from Tables 1-3 may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes of Tables 1-3 are compared to the expression levels of those same genes exposed to a known toxin alone. Compounds that modulate the expression patterns of the known toxin(s) would be expected to modulate potential toxic physiological effects in vivo. The genes in Tables 1-3 are particularly appropriate marks in these assays as they are differentially expressed in cells upon exposure to a known hepatotoxin.

In another format, cell lines that contain reporter gene fusions between the open reading frame and/or the transcriptional regulatory regions of a gene in Tables 1-3 and any assayable fusion partner may be prepared. Numerous assayable fusion partners are known and readily available including the firefly luciferase gene and the gene encoding chloramphenicol acetyltransferase (Alam et al. (1990) Anal. Biochem. 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Tables 1-3. For instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al. (Molecular Cloning: A Laboratory Manual, 2nd Ed. Cold Spring Harbor Laboratory Press, 1989).

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines may be transduced or transfected with an expression vehicle (e.g., a plasmid or viral vector) construct comprising an operable non-translated 5′-promoter containing end of the structural gene encoding the gene products of Tables 1-3 fused to one or more antigenic fragments or other detectable markers, which are peculiar to the instant gene products, wherein said fragments are under the transcriptional control of said promoter and are expressed as polypeptides whose molecular weight can be distinguished from the naturally occurring polypeptides or may further comprise an immunologically distinct or other detectable tag. Such a process is well known in the art (see Maniatis).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample is then compared with the control samples (no exposure and exposure to a known toxin) where only the excipient is contacted with the cells and an increase or decrease in the immunologically generated signal from the “agent-contacted” sample compared to the control is used to distinguish the effectiveness and/or toxic effects of the agent.

Another embodiment of the present invention provides methods for identifying agents that modulate at least one activity of a protein(s) encoded by the genes in Tables 1-3. Such methods or assays may utilize any means of monitoring or detecting the desired activity.

In one format, the relative amounts of a protein (Tables 1-3) between a cell population that has been exposed to the agent to be tested compared to an un-exposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant G A. in: Meyers (ed.) Molecular Biology and Biotechnology (New York, VCH Publishers, 1995), pp. 659-664). A skilled artisan can readily recognize that there is no limit as to the structural nature of the agents of the present invention.

### Nucleic Acid Assay Formats

The genes identified as being differentially expressed upon exposure to a known hepatotoxin (Tables 1-3) may be used in a variety of nucleic acid detection assays to detect or quantititate the expression level of a gene or multiple genes in a given sample. The genes described in Tables 1-3 may also be used in combination with one or more additional genes whose differential expression is associate with toxicity in a cell or tissue. In preferred embodiments, the genes in Tables 1-3 may be combined with one or more of the genes described in related applications 60/222,040, 60/244,880, 60/290,029, 60/290,645, 60/292,336, 60/295,798, 60/297,457, 60/298,884 and 60/303,459, all of which are incorporated by reference on page 1 of this application.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, amplification based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/11755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be on the order of about a square centimeter. Probes corresponding to the genes of Tables 1-3 or from the related applications described above may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al., Nat. Biotechnol. (1996) 14, 1675-1680; McGall et al., Proc. Nat. Acad. Sci. USA (1996) 93, 13555-13460). Such probe arrays may contain at least two or more oligonucleotides that are complementary to or hybridize to two or more of the genes described in Tables 1-3. For instance, such arrays may contain oligonucleotides that are complementary or hybridize to at least 2, 3, 4, 5, 6, 7, 8, 9, 10, 20, 30, 50, 70, 100 or more the genes described herein. Preferred arrays contain all or nearly all of the genes listed in Tables 1-3, or individually, the gene sets of Tables 3A-3S. In a preferred embodiment, arrays are constructed that contain oligonucleotides to detect all or nearly all of the genes in any one of or all of Tables 1-3 on a single solid support substrate, such as a chip.

The sequences of the expression marker genes of Tables 1-3 are in the public databases. Table 1 provides the GenBank Accession Number for each of the sequences (see www.ncbi.nlm.nih.gov/). The sequences of the genes in GenBank are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate. These sequences may be used in the methods of the invention or may be used to produce the probes and arrays of the invention. In some embodiments, the genes in Tables 1-3 that correspond to the genes or fragments previously associated with a toxic response may be excluded from the Tables.

As described above, in addition to the sequences of the GenBank Accessions Numbers disclosed in the Tables 1-3, sequences such as naturally occurring variant or polymorphic sequences may be used in the methods and compositions of the invention. For instance, expression levels of various allelic or homologous forms of a gene disclosed in the Tables 1-3 may be assayed. Any and all nucleotide variations that do not alter the functional activity of a gene listed in the Tables 1-3, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes described in Tables 1-3 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

### Probe Design

One of skill in the art will appreciate that an enormous number of array designs are suitable for the practice of this invention. The high density array will typically include a number of test probes that specifically hybridize to the sequences of interest. Probes may be produced from any region of the genes identified in the Tables and the attached representative sequence listing. In instances where the gene reference in the Tables is an EST, probes may be designed from that sequence or from other regions of the corresponding full-length transcript that may be available in any of the sequence databases, such as those herein described. See WO99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are 20 or 25 nucleotides in length. In another preferred embodiment, test probes are double or single strand DNA sequences. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent) Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene in the accompanying Tables 1-3. The difference in intensity between the perfect match and the mismatch probe provides a good measure of the concentration of the hybridized material.

### Nucleic Acid Samples

Cell or tissue samples may be exposed to the test agent in vitro or in vivo. When cultured cells or tissues are used, appropriate mammalian liver extracts may also be added with the test agent to evaluate agents that may require biotransformation to exhibit toxicity.

In a preferred format, primary isolates of animal or human hepatocytes which already express the appropriate complement of drug-metabolizing enzymes may be exposed to the test agent without the addition of mammalian liver extracts.

The genes which are assayed according to the present invention are typically in the form of mRNA or reverse transcribed mRNA. The genes may be cloned or not. The genes may be amplified or not. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I Theory and Nucleic Acid Preparation, P. Tijssen, Ed., Elsevier, N.Y. (1993). Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

### Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling. See Pirrung, U.S. Pat. No. 5,143,854.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO93/09668 and WO01/23614. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

### Hybridization

Nucleic acid hybridization simply involves contacting a probe and target nucleic acid under conditions where the probe and its complementary target can form stable hybrid duplexes through complementary base pairing. See WO99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6×SSPET at 37° C. (0.005% Triton X-100), to ensure hybridization and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

### Signal Detection

The hybridized nucleic acids are typically detected by detecting one or more labels attached to the sample nucleic acids. The labels may be incorporated by any of a number of means well known to those of skill in the art. See WO99/32660.

### Databases

The present invention includes relational databases containing sequence information, for instance, for the genes of Tables 1-3, as well as gene expression information from tissue or cells exposed to various standard toxins, such as those herein described (see Table 3A-3S). Databases may also contain information associated with a given sequence or tissue sample such as descriptive information about the gene associated with the sequence information (see Table 1), or descriptive information concerning the clinical status of the tissue sample, or the animal from which the sample was derived. The database may be designed to include different parts, for instance a sequence database and a gene expression database. Methods for the configuration and construction of such databases are widely available, for instance, see U.S. Pat. No. 5,953,727, which is herein incorporated by reference in its entirety.

The databases of the invention may be linked to an outside or external database such as GenBank (www.ncbi.nlm.nih.gov/entrez.index.html); KEGG (www.genome.ad.jp/kegg); SPAD (www.grt.kyushu-u.ac.jp/spad/index.html); HUGO (www.gene.ucl.ac.uk/hugo); Swiss-Prot (www.expasy.ch.sprot); Prosite (www.expasy.ch/tools/scnpsit1.html); OMIM (www.ncbi.nlm.nih.gov/omim); GDB (www.gdb.org); and GeneCard (bioinformatics.weizmann.ac.il/cards). In a preferred embodiment, as described in Tables 1-3, the external database is GenBank and the associated databases maintained by the National Center for Biotechnology Information (NCBI) (www.ncbi.nlm.nih.gov).

Any appropriate computer platform may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising one or more of the genes in Tables 1-3, comprising the step of comparing the expression level of at least one gene in Tables 1-3 in a cell or tissue exposed to a test agent to the level of expression of the gene in the database. Such methods may be used to predict the toxic potential of a given compound by comparing the level of expression of a gene or genes in Tables 1-3 from a tissue or cell sample exposed to the test agent to the expression levels found in a control tissue or cell samples exposed to a standard toxin or hepatotoxin such as those herein described. Such methods may also be used in the drug or agent screening assays as described below.

### Kits

The invention further includes kits combining, in different combinations, high-density oligonucleotide arrays, reagents for use with the arrays, protein reagents encoded by the genes of the Tables, signal detection and array-processing instruments, gene expression databases and analysis and database management software described above. The kits may be used, for example, to predict or model the toxic response of a test compound, to monitor the progression of hepatic disease states, to identify genes that show promise as new drug targets and to screen known and newly designed drugs as discussed above.

The databases packaged with the kits are a compilation of expression patterns from human or laboratory animal genes and gene fragments (corresponding to the genes of Tables 1-3). In particular, the database software and packaged information include the expression results of Tables 1-3 that can be used to predict toxicity of a test agent by comparing the expression levels of the genes of Tables 1-3 induced by the test agent to the expression levels presented in Tables 3A-3S. In another format, database and software information may be provided in a remote electronic format, such as a website, the address of which may be packaged in the kit.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with use with microarrays is discussed in Balaban et al., U.S. Pat. No. 6,229,911, a computer-implemented method for managing information, stored as indexed Tables 1-3, collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, disclose a software-based method for identifying mutations in a nucleic acid sequence based on differences in probe fluorescence intensities between wild type and mutant sequences that hybridize to reference sequences.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Toxicity Markers

The hepatotoxins amitryptiline, ANIT, acetaminophen, carbon tetrachloride, CPA, diclofenac, estradiol, indomethacin, valproate, WY-14643 and control compositions were administered to male Sprague-Dawley rats at various time points using administration diluents, protocols and dosing regimes as previously described in the art and previously described in the priority applications discussed above.

After administration, the dosed animals were observed and tissues were collected as described below:

OBSERVATION OF ANIMALS

| | | |
|---|---|---|
| 1. | Clinical | Twice daily - mortality and moribundity check. |
| | Observations | Cage Side Observations - skin and fur, eyes and |
| | | mucous membrane, respiratory system, circulatory |
| | | system, autonomic and central nervous system, |
| | | somatomotor pattern, and behavior pattern. |
| | | Potential signs of toxicity, including tremors, |
| | | convulsions, salivation, diarrhea, lethargy, coma |
| | | or other atypical behavior or appearance, were |
| | | recorded as they occurred and included a time |
| | | of onset, degree, and duration. |
| 2. | Physical | Prior to randomization, prior to initial treatment, |
| | Examinations | and prior to sacrifice. |
| 3. | Body Weights | Prior to randomization, prior to initial treatment, |
| | | and prior to sacrifice. |

CLINICAL PATHOLOGY

| | | |
|---|---|---|
| 1. | Frequency | Prior to necropsy. |
| 2. | Number of animals | All surviving animals. |
| 3. | Bleeding | Blood was obtained by puncture of the |
| | Procedure | orbital sinus while under 70% CO₂/30% O₂ |
| | | anesthesia. |
| 4. | Collection of | Approximately 0.5 mL of blood was |
| | Blood Samples | collected into EDTA tubes for evaluation |
| | | of hematology parameters. Approximately |
| | | 1 mL of blood was collected into serum |
| | | separator tubes for clinical chemistry |
| | | analysis. Approximately 200 uL of plasma |
| | | was obtained and frozen at ^{~}−80° |
| | | C. for test compound/metabolite |
| | | estimation. An additional ^{~}2 mL of blood |
| | | was collected into a 15 mL conical poly- |
| | | propylene vial to which ^{~}3 mL of Trizol |
| | | was immediately added. The contents |
| | | were immediately mixed with a vortex and |
| | | by repeated inversion. The tubes were |
| | | frozen in liquid nitrogen and stored |
| | | at ^{~}−80° C. |

### Termination Procedures

Terminal Sacrifice
Approximately 1 and 3 and 6 and 24 and 48 hours and 5-7 days after the initial dose, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.
Necropsies were conducted on each animal following procedures approved by board-certified pathologists.
Animals not surviving until terminal sacrifice were discarded without necropsy (following euthanasia by carbon dioxide asphyxiation, if moribund). The approximate time of death for moribund or found dead animals was recorded.

Postmortem Procedures
Fresh and sterile disposable instruments were used to collect tissues. Gloves were worn at all times when handling tissues or vials. All tissues were collected and frozen within approximately 5 minutes of the animal's death. The liver sections and kidneys were frozen within approximately 3-5 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing
Liver
1. Right medial lobe—snap frozen in liquid nitrogen and stored at ˜−80° C.
2. Left medial lobe—Preserved in 10% neutral-buffered formalin (NBF) and evaluated for gross and microscopic pathology.
3. Left lateral lobe—snap frozen in liquid nitrogen and stored at ˜−80° C.
Heart
A sagittal cross-section containing portions of the two atria and of the two ventricles was preserved in 10% NBF. The remaining heart was frozen in liquid nitrogen and stored at ˜−80° C.
Kidneys (both)
1. Left—Hemi-dissected; half was preserved in 10% NBF and the remaining half was frozen in liquid nitrogen and stored at ˜−80° C.
2. Right—Hemi-dissected; half was preserved in 10% NBF and the remaining half was frozen in liquid nitrogen and stored at ˜−80° C.
Testes (both)
A sagittal cross-section of each testis was preserved in 10% NBF. The remaining testes were frozen together in liquid nitrogen and stored at ˜−80° C.
Brain (whole)
A cross-section of the cerebral hemispheres and of the diencephalon was preserved in 10% NBF, and the rest of the brain was frozen in liquid nitrogen and stored at ˜−80° C.

Microarray sample preparation was conducted with minor modifications, following the protocols set forth in the Affymetrix GeneChip Expression Analysis Manual. Frozen tissue was ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg tissue weight. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The cDNA was phenol-chloroform extracted and ethanol precipitated to a final concentration of 1 μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip® version 3.0 and Expression Data Mining (EDMT) software (version 1.0), GeneExpress2000, and S-Plus.

Table 1 discloses those genes that are differentially expressed upon exposure to the named toxins and their corresponding GenBank Accession and Sequence Identification numbers, the identities of the metabolic pathways in which the genes function, the gene names if known, and the unigene cluster titles. The comparison code represents the various toxicity or liver pathology state that each gene is able to discriminate as well as the individual toxin type associated with each gene. The codes are defined in Table 2. The GLGC ID is the internal Gene Logic identification number.

Table 2 defines the comparison codes used in Table 1.

Tables 3A-3S disclose the summary statistics for each of the comparisons performed. Each gene is identified by its Gene Logic identification number and can be cross-referenced to a gene name and representative SEQ ID NO. in Table 1. The group mean (e.g. toxicity group) is the mean signal intensity as normalized for the various chip parameters in the samples that are being assayed for in the particular comparison. The non-group (e.g. non-toxicity group) mean represents the mean signal intensity as normalized for the various chip parameters in the samples that are not being assayed for in the particular comparison. The mean values are derived from Average Difference (AveDiff) values for a particular gene, averaged across the corresponding samples. Each individual Average Difference value is calculated by integrating the intensity information from multiple probe pairs that are tiled for a particular fragment. The normalization algorithm used to calculate the AveDiff is based on the observation that the expression intensity values from a single chip experiment have different distributions, depending on whether small or large expression values are considered. Small values, which are assumed to be mostly noise, are approximately normally distributed with mean zero, while larger values roughly obey a log-normal distribution; that is, their logarithms are normally distributed with some nonzero mean.

The normalization process computes separate scale factors for “non-expressors” (small values) and “expressors” (large ones). The inputs to the algorithm are pre-normalized Average Difference values, which are already scaled to set the trimmed mean equal to 100. The algorithm computes the standard deviation SD noise of the negative values, which are assumed to come from non-expressors. It then multiplies all negative values, as well as all positive values less than 2.0*SD noise, by a scale factor proportional to 1/SD noise.

Values greater than 2.0*SD noise are assumed to come from expressors. For these values, the standard deviation SD log(signal) of the logarithms is calculated. The logarithms are then multiplied by a scale factor proportional to 1/SD log(signal) and exponentiated. The resulting values are then multiplied by another scale factor, chosen so there will be no discontinuity in the normalized values from unscaled values on either side of 2.0*SD noise. Some AveDiff values may be negative due to the general noise involved in nucleic acid hybridization experiments. Although many conclusions can be made corresponding to a negative value on the GeneChip platform, it is difficult to assess the meaning behind the negative value for individual fragments. Our observations show that, although negative values are observed at times within the predictive gene set, these values reflect a real biological phenomenon that is highly reproducible across all the samples from which the measurement was taken. For this reason, those genes that exhibit a negative value are included in the predictive set. It should be noted that other platforms of gene expression measurement may be able to resolve the negative numbers for the corresponding genes. The predictive ability of each of those genes should extend across platforms, however. Each mean value is accompanied by the standard deviation for the mean. LDA is the linear discriminant analysis that measures the ability of each gene to predict whether or not a sample is toxic. The LDA score is calculated by the following steps:

### Calculation of a Discriminant Score.

Let Xᵢ represent the AveDiff values for a given gene across the Group I samples, i=1 . . . n.

Let Yᵢ represent the AveDiff values for a given gene across the Group 2 samples, i=1 . . . t.

The calculations proceed as follows:
1. Calculate mean and standard deviation for Xᵢ's and Yᵢ's, and denote these by m_{X}, m_{Y}, s_{X},s_{Y}.
2. For all Xᵢ's and Yᵢ's, evaluate the function f(z)=((1/s_{Y})*exp(−0.5*((z−m_{Y})/s_{Y})²))/(((1/s_{Y})*exp(−0.5*((z−m_{Y})/s_{Y})²))+((1/s_{X})*exp(−0.5*((z−m_{X})/s_{X})²))).
3. The number of correct predictions, say P, is then the number of Yᵢ's such that f(Yᵢ)>0.5 plus the number of Xᵢ's such that f(Xᵢ)<0.5.
4. The discriminant score is then P/(n+t)

Linear discriminant analysis uses both the individual measurements of each gene and the calculated measurements of all combinations of genes to classify samples. For each gene a weight is derived from the mean and standard deviation of the tox and nontox groups. Every gene is multiplied by a weight and the sum of these values results in a collective discriminate score. This discriminant score is then compared against collective centroids of the tox and nontox groups. These centroids are the average of all tox and nontox samples respectively. Therefore, each gene contributes to the overall prediction. This contribution is dependent on weights that are large positive or negative numbers if the relative distances between the tox and nontox samples for that gene are large and small numbers if the relative distances are small. The discriminant score for each unknown sample and centroid values can be used to calculate a probability between zero and one as to which group the unknown sample belongs.

### Example 2

### General Toxicity Modeling

Samples were selected for grouping into tox-responding and non-tox-responding groups by examining each study individually with PCA to determine which treatments had an observable response. Only groups where confidence of their tox-responding and non-tox-responding status was established were included in building a general tox model.

Two general types of models were built for general toxicity determination. One model used information from the expression patterns of each gene individually and then combined all the information using linear weights for each gene. The second type determined orthogonal vectors describing all the expression information collectively and used these composite vectors to predict toxicity.

Over 500 linear discriminant models were generated to describe toxic and non-toxic samples. The top 10, 25, 50 and 100 discriminant genes were used to determine toxicity by calculating each gene's contribution with homo and heteroscedastic treatment of variance and inclusion or exclusion of mutual information between genes. Prediction of samples within the database exceeded 90% for most models. In addition, models were built by sequential use of two, five, ten, twenty five, and fifty genes, starting with the best discriminators and proceeding to the worst discriminators without replication. All discriminating genes and/or ESTs had at least 70% discriminate ability, which was previously determined to be significant via randomization experiments. It was determined that combinations of genes generally provided a better predictive ability then individual genes and that the more genes used the better predictive ability. It was also determined that combining the worst fifty discriminating genes provided better prediction than the best single gene and that many combinations of two or more genes provided better prediction than the best individual gene. Although the preferred embodiment includes fifty or more genes, many pairings or greater combinations of genes can work better than individual genes. All combinations of two or more genes from the selected list may be used to predict toxicity. These combinations could be selected by pairing in an ordered, agglomerate, divisive, or random approach. Further, as yet undetermined genes could be combined with individual or combination of genes described here to increase predictive ability. However, the genes described here may contribute most of the predictive ability of any such undetermined combinations.

The second approach used has been described in U.S. Provisional Application 60/______, using this approach all 527 genes and/or EST were used to predict toxic from non-toxic samples with greater than 94% accuracy when 15 components are used. Although using the first fifteen components provided a preferred model, other variations of this method can provide adequate predictive ability. These include selective inclusion of components via agglomerate, divisive, or random approaches or extraction of loading and combining them in ordered, agglomerate, divisive, or random approaches. Also the use of these composite variables in logistic regression to determine classification of samples can also be accomplished with linear discriminate analysis, neural or Bayesian networks, or other forms of regression and classification based on categorical or continual dependent and independent variables.

### Example 3

### Modeling Methods

The above modeling methods provide broad approaches of combining the expression of genes to predict sample toxicity. One method uses each variable individually and weights them; the other combines variables as a composite measure and adds weights to them after combination into a new variable. One could also provide no weight in a simple voting method or determine weights in a supervised or unsupervised method using agglomerate, divisive, or random approaches. All or selected combinations of genes may be combined in ordered, agglomerate, or divisive, supervised or unsupervised clustering algorithms with unknown samples for classification. Any form of correlation matrix may also be used to classify unknown samples. The spread of the group distribution and discriminate score alone provide enough information to enable a skilled person to generate all of the above types of models with accuracy that can exceed discriminate ability of individual genes. Some examples of methods that could be used individually or in combination after transformation of data types include but are not limited to: Discriminant Analysis, Multiple Discriminant Analysis, logistic regression, multiple regression analysis, linear regression analysis, conjoint analysis, canonical correlation, hierarchical cluster analysis, k-means cluster analysis, self-organizing maps, multidimensional scaling, structural equation modeling, support vector machine determined boundaries, factor analysis, neural networks, bayesian classifications, and resampling methods.

### Example 4

### Grouping of Individual compound and Pathology Classes

Samples were grouped into individual pathology classes based on known toxicological responses and observed clinical chemical and pathology measurements or into early and late phases of observable toxicity within a compound (Tables 3A-3S). The top 10, 25, 50, 100 genes based on individual discriminate scores were used in a model to ensure that combination of genes provided a better prediction than individual genes. As described above, all combinations of two or more genes from this list could potentially provide better prediction than individual genes when selected in any order or by ordered, agglomerate, divisive, or random approaches. In addition, combining these genes with other genes could provide better predictive ability, but most of this predictive ability would come from the genes listed here.

Samples may be considered toxic if they score positive in any pathological or individual compound class represented here or in any modeling method mentioned under general toxicology models based on combination of individual time and dose grouping of individual toxic compounds obtainable from the data. The pathological groupings and early and late phase models are preferred examples of all obtainable combinations of sample time and dose points. Most logical groupings with one or more genes and one or more sample dose and time points should produce better predictions of general toxicity, pathological specific toxicity, or similarity to known toxicant than individual genes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1-54. (canceled)

55. A method of predicting for the hepatoxicity of a test compound, comprising:
(a) preparing a gene expression profile of at least ten genes from a liver tissue or liver cell sample exposed to the test compound; and
(b) comparing the expression levels of said genes from the gene expression profile to a database comprising the gene expression levels of said genes derived from liver tissue or liver cell samples that have been exposed to at least one known hepatotoxin, wherein said at least ten genes are selected from the genes and ESTs in any one of Tables 3A-3S, thereby predicting for the hepatoxicity of the test compound.

56. The method of claim 55, wherein the gene expression profile prepared from the liver tissue or liver cell sample comprises the level of expression for at least 100 genes.

57. The method of claim 55, wherein expression levels for said at least ten genes from the gene expression profile are compared to Toxic Mean and/or NonToxic Mean values in a database comprising any one of Tables 3A-3S.

58. The method of claim 57, wherein the level of expression for each gene is normalized prior to comparison.

59. The method of claim 55, wherein the database comprises all of the data in any one of Tables 3A-3S.

60. The method of claim 55, wherein the expression levels of at least 15 genes are compared to the database.

61. The method of claim 55, wherein the expression levels of at least 20 genes are compared to the database.

62. The method of claim 55, wherein the expression levels of at least 25 genes are compared to the database.

63. The method of claim 55, wherein the expression levels of at least 30 genes are compared to the database.

64. The method of claim 55, wherein the expression levels of at least 50 genes are compared to the database.

65. The method of claim 55 wherein the expression levels of at least 75 genes are compared to the database.

66. The method of claim 55, wherein the expression levels of at least 100 genes are compared to the database.

67. The method of claim 55, wherein the liver cell or liver tissue sample is exposed to the test compound in vivo and the liver cell or liver tissue samples from which database information is derived are exposed to the at least one known hepatotoxin in vivo.

68. The method of claim 67, wherein the hepatoxicity is associated with at least one liver disease pathology selected from the group consisting of liver damage induced by hepatitis, liver damage induced by NSAIDS, liver necrosis with fatty liver, liver necrosis without fatty liver and liver damage induced by compounds that form protein adducts.

69. The method of claim 55, wherein the hepatotoxin is selected from the group consisting of amitryptiline, ANIT, acetaminophen, carbon tetrachloride, cyproterone acetate, diclofenac, estradiol, indomethacin, valproate, and WY-14643.

70. The method of claim 55, wherein the gene expression profile is produced by hybridization of nucleic acids to a microarray.

71. The method of claim 55, wherein the liver cell or liver tissue sample is a rat liver cell or rat liver tissue sample.

72. The method of claim 55, wherein said selected genes are rat genes.

73. The method of claim 67, wherein the hepatoxicity is liver necrosis.

74. A method of predicting for the liver toxicity of a test compound, comprising:
(a) preparing a gene expression profile of at least ten genes from a liver tissue or liver cell sample exposed to the test compound; and
(b) comparing the expression levels of said genes from the gene expression profile to a database comprising the gene expression levels of said genes derived from liver tissue or liver cell samples that have been exposed to at least one known liver toxin, wherein said at least ten genes are selected from the genes and ESTs in any one of Tables 3A-3S, thereby predicting for the liver toxicity of the test compound.

75. The method of claim 74, wherein the gene expression profile prepared from the liver tissue or liver cell sample comprises the level of expression for at least 100 genes.

76. The method of claim 74, wherein expression levels for said at least ten genes from the gene expression profile are compared to Toxic Mean and/or NonToxic Mean values in a database comprising Tables 3A-3S.

77. The method of claim 74, wherein the level of expression for each gene is normalized prior to comparison.

78. The method of claim 74, wherein the database comprises all of the data in any one of Tables 3A-3S.

79. The method of claim 74, wherein the expression levels of at least 15 genes are compared to the database.

80. The method of claim 74, wherein the expression levels of at least 20 genes are compared to the database.

81. The method of claim 74, wherein the expression levels of at least 25 genes are compared to the database.

82. The method of claim 74, wherein the expression levels of at least 30 genes are compared to the database.

83. The method of claim 74, wherein the expression levels of at least 50 genes are compared to the database.

84. The method of claim 74, wherein the expression levels of at least 75 genes are compared to the database.

85. The method of claim 74, wherein the expression levels of at least 100 genes are compared to the database.

86. The method of claim 74 wherein the liver cell or liver tissue sample is exposed to the compound in vivo and the liver cell or liver tissue samples from which database information is derived are exposed to the at least one known liver toxin in vivo.

87. The method of claim 86, wherein the liver toxicity is associated with at least one liver disease pathology selected from the group consisting of liver damage induced by hepatitis, liver damage induced by NSAIDS, liver necrosis with fatty liver, liver necrosis without fatty liver and liver damage induced by compounds that form protein adducts.

88. The method of claim 86, wherein the liver toxin is selected from the group consisting of amitryptiline, ANIT, acetaminophen, carbon tetrachloride, cyproterone acetate, diclofenac, estradiol, indomethacin, valproate, and WY-14643.

89. The method of claim 74, wherein the gene expression profile is produced by hybridization of nucleic acids to a microarray.

90. The method of claim 74, wherein the liver cell or liver tissue sample is a rat liver cell or rat liver tissue sample.

91. The method of claim 74, wherein said selected genes are rat genes.

92. The method of claim 86 wherein the liver toxicity is liver necrosis.
